(19) United States
(12) Patent Application Publication
(10) Pub. No.: US 2011/0171437 A1
(43) Pub. Date: Jul. 14, 2011

(54) Phthalocyanines and Their Use in Ink-Jet Printing

(76) Inventor: Prakash Patel, Manchester (GB)

(21) Appl. No.: 13/060,410

(22) PCT Filed: Sep. 22, 2009
(86) PCT No.: PCT/GB2009/051228

§ 371 Date: Feb. 23, 2011

(30) Foreign Application Priority Data
Oct. 1, 2008 (GB) 0817932.7

(51) Int. Cl. B32B 3/10; C07D 251/54; C09D 11/02; B41J 2/015; B41J 2/175

(52) U.S. Cl. 428/195.1; 544/197; 106/31.78; 347/20; 347/86

(57) ABSTRACT

A mixture of compounds of Formula (1) and salts thereof:

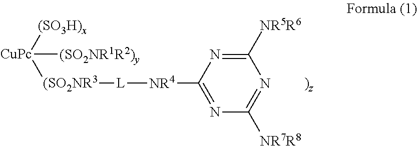

wherein:
Pc represents a phthalocyanine nucleus;
R¹ is H or optionally substituted C₁₋₈alkyl;
R², R³, R⁴, R⁵ and R⁷ are each independently H or methyl;
R⁶ is H, methyl or aryl carrying 1 to 3 carboxy groups and optionally other substituents;
R⁸ is aryl, carrying 1 to 3 carboxy groups and optionally other substituents;
L is optionally substituted C₁₋₄alkylene;
x, y, and z are each independently greater than 0 and less than 4;
x+y+z is in the range of 2 to 4; and
provided that:
the substituents represented by x, y and z are attached to the phthalocyanine nucleus by a β-position; and
L, R¹, R⁶ and R⁸ do not carry a sulfonic acid substituent.
   Also compositions, novel amines, printed material and ink-jet cartridges.

This invention relates to compounds, compositions and inks, to printing processes, to printed substrates and to ink-jet printer cartridges.

Ink-jet printing is a non-impact printing technique in which droplets of ink are ejected through a fine nozzle onto a substrate without bringing the nozzle into contact with the substrate. The set of inks used in this technique typically comprise yellow, magenta, cyan and black inks.

With the advent of high-resolution digital cameras and ink-jet printers it is becoming increasingly common for consumers to print photographs using an ink-jet printer.

While ink-jet printers have many advantages over other forms of printing and image development there are still technical challenges to be addressed. For example, there are the contradictory requirements of providing ink colorants that are soluble in the ink medium and yet display excellent wet-fastness (i.e. prints do not run or smudge when printed). The inks also need to dry quickly to avoid sheets sticking together after they have been printed, but they should not crust over the tiny nozzles used in the printer. Storage stability is also important to avoid particle formation that could block the printer-head nozzles especially since consumers can keep an ink-jet ink cartridge for several months. Furthermore, and especially important with photographic quality reproductions, the resultant images should not bronze or fade rapidly on exposure to light or common oxidising gases such as ozone. It is also important that the shade and chroma of the colorant are exactly right so that an image may be optimally reproduced.

Most cyan colorants used in ink-jet printing are based on phthalocyanines and problems of fading and shade change on exposure to light and contact with ozone are particularly acute with dyes of this class.

Thus developing new colorants for ink-jet printing presents a unique challenge in balancing all these conflicting and demanding properties.

The present invention provides a mixture of compounds of Formula (1) and salts thereof:

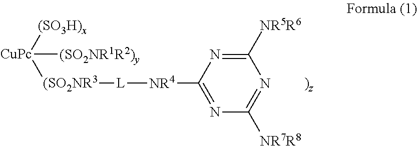

wherein:
Pc represents a phthalocyanine nucleus of formula;

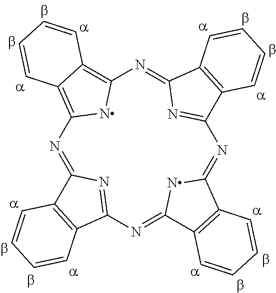

R¹ is H or optionally substituted C₁₋₈alkyl;
R², R³, R⁴, R⁵ and R⁷ are each independently H or methyl;
R⁶ is H, methyl or aryl carrying 1 to 3 carboxy groups and optionally other substituents;
R⁸ is aryl carrying 1 to 3 carboxy groups and optionally other substituents;
L is optionally substituted C₁₋₄alkylene;
x, y, and z are each independently greater than 0 and less than 4;
x+y+z is in the range of 2 to 4; and
provided that:
the substituents represented by x, y and z are attached to the phthalocyanine nucleus by a β-position; and
L, R¹, R⁶ and R⁸ do not carry a sulfonic acid substituent.

Preferably R¹ is H; methyl; optionally substituted C₁₋₄alkyl carrying at least one water solubilising substituent preferably selected from the group consisting of: —OH, —CO₂H and —PO₃H₂; C₁₋₄alkyl with a phenyl substituent carrying 1 or 2, particularly 2, water solubilising groups, especially water solubilising groups selected from —CO₂H and —PO₃H₂ and more especially —CO₂H; or C₁₋₈alkyl substituted with 2 or more, preferably 4 or more, —OH groups and optionally other substituents, especially substituents selected from the group consisting of —CO₂H, and —PO₃H₂.

Preferably R⁶ is H, methyl or optionally substituted aryl carrying 1 or 2 carboxy groups. More preferably R⁶ is H, methyl or optionally substituted phenyl carrying 1 or 2 carboxy groups, especially H, methyl or phenyl carrying 2 carboxy groups.

Preferably R⁸ is optionally substituted aryl carrying 1 or 2 carboxy groups. More preferably R⁸ is optionally substituted phenyl carrying 1 or 2 carboxy groups, especially phenyl carrying 2 carboxy groups.

In one preferred embodiment R¹, R², R³, R⁴, R⁵, R⁶ and R⁷ are all H.

Preferably L is unsubstituted C₁₋₄alkylene, especially —C₂H₄—.

Preferred optional substituents which may be present on any one of L, R¹, R⁶ and R⁸ are independently selected from: optionally substituted alkoxy (preferably C₁₋₄-alkoxy), optionally substituted aryl (preferably phenyl), optionally substituted aryloxy (preferably phenoxy), optionally substituted heterocyclyl, polyalkylene oxide (preferably polyethylene oxide or polypropylene oxide), phosphato, nitro, cyano, halo, ureido, hydroxy, ester, —NR^{a}R^{b}, —COR^{a}, —CONR^{a}R^{b}, —NHCOR^{a}, carboxyester, sulfone, and —SO₂NR^{a}R^{b}, wherein R^{a} and R^{b} are each independently H or optionally substituted alkyl (especially C₁₋₄-alkyl). R⁶ (when it is aryl) and R⁸ may also carry an optionally substituted alkyl (especially C₁₋₄-alkyl) substituent. Optional substituents for any of the substituents described above may be selected from the same list of substituents.

Preferably R⁶ (when it is aryl) and R⁸ carry no substituents other than the carboxy groups.

Preferably x is 0.1 to 3, more preferably 0.2 to 2.0.

Preferably y is 0.1 to 3, more preferably 0.2 to 2.0.

Preferably z is 0.1 to 3.5, more preferably 0.5 to 3.0, especially 0.8 to 3.0 and more especially 1.0 to 3.0.

In one preferred embodiment x is greater than 1.

In another preferred embodiment y is less than 1 and preferably less than 0.5.

In a third preferred embodiment z is less than 1 and preferably less than 0.3.

In a fourth preferred embodiment z is greater than 3 and more preferably greater than 3.5.

The sum of (x+y+z) is preferably 3 to 4, more preferably the sum of (x+y+z) is 4.

The compounds of Formula (1) are also preferably free from fibre reactive groups. The term fibre reactive group is well known in the art and is described for example in EP 0356014 A1. Fibre reactive groups are capable, under suitable conditions, of reacting with the hydroxyl groups present in cellulosic fibres or with the amino groups present in natural fibres to form a covalent linkage between the fibre and the dye. As examples of fibre reactive groups excluded from the compounds of Formula (1) there may be mentioned aliphatic sulfonyl groups which contain a sulfate ester group in beta-position to the sulfur atom, e.g. beta-sulfato-ethylsulfonyl groups, alpha, beta-unsaturated acyl radicals of aliphatic carboxylic acids, for example acrylic acid, alpha-chloro-acrylic acid, alpha-bromoacrylic acid, propiolic acid, maleic acid and mono- and dichloro maleic; also the acyl radicals of acids which contain a substituent which reacts with cellulose in the presence of an alkali, e.g. the radical of a halogenated aliphatic acid such as chloroacetic acid, beta-chloro and beta-bromopropionic acids and alpha, beta-dichloro- and dibromopropionic acids or radicals of vinylsulfonyl- or beta-chloroethylsulfonyl- or beta-sulfatoethyl-sulfonyl-endo-methylene cyclohexane carboxylic acids. Other examples of cellulose reactive groups are tetrafluorocyclobutyl carbonyl, trifluoro-cyclobutenyl carbonyl, tetrafluorocyclobutylethenyl carbonyl, trifluoro-cyclobutenylethenyl carbonyl; activated halogenated 1,3-dicyanobenzene radicals; and heterocyclic radicals which contain 1, 2 or 3 nitrogen atoms in the heterocyclic ring and at least one cellulose reactive substituent on a carbon atom of the ring, for example a triazinyl halide.

Acid or basic groups on the compounds of Formula (1), particularly acid groups, are preferably in the form of a salt. Thus, all Formulae shown herein include the compounds in salt form.

Preferred salts are alkali metal salts, especially lithium, sodium and potassium, ammonium and substituted ammonium salts (including quaternary amines such as ((CH₃)₄N⁺) and mixtures thereof. Especially preferred are salts with sodium, lithium, ammonia and volatile amines, more especially sodium salts. Compounds of Formula (1) may be converted into a salt using known techniques.

The compounds of Formula (1) may exist in tautomeric forms other than those shown in this specification. These tautomers are included within the scope of the present invention.

The mixtures of compounds of Formula (1) may be prepared by any method known in the art, and particularly by any method which comprises cyclisation of appropriate β-substituted phthalic acid, phthalonitrile, iminoisoindoline, phthalic anhydride, phthalimide or phthalamide in the presence of a suitable nitrogen source (if required), a metal salt, such as CuCl₂, (if required) and a base such as 1,8-diazabicyclo[5.4.0]undec-7-ene (DBU).

When the substituent represented by z comprises a sulfonamide linking group then preferably mixtures of compounds of Formula (1) may be prepared by cyclisation of 4-sulfo-phthalic acid in the presence of a nitrogen source, such as urea, a metal salt, such as CuCl₂, (if required) and a base such as 1,8-diazabicyclo[5.4.0]undec-7-ene (DBU) to give phthalocyanine β-tetrasulfonic acid. The phthalocyanine β-tetrasulfonic acid may then be chlorinated and the sulfonyl chloride groups so formed are reacted with compounds of formula HNR¹R² and amines of Formula (2) as described below where R¹ and R² are as hereinbefore defined. This reaction is preferably performed in water at a pH above 7. Typically the reaction is performed at a temperature of 30 to 70° C. and is usually complete in less than 24 hours. The compound of formula HNR¹R² and amine of Formula (2) may be used as a mixture or added sequentially.

Many of the compounds of formula HNR¹R² are commercially available, for example ammonia, others may be made easily by a skilled person using methods which are well known in the art.

The ratio of sulfo to sulfonamide substituents may be varied by varying the nature and amount of chlorinating agent used, the relative amounts of the compound of formula HNR¹R² and amine of Formula (2) used and the reaction conditions in both reactions.

A skilled person will appreciate that the product of these reactions will be a disperse mixture and so the values of x, y and z will represent an average of the groups present in the mixture.

When phthalocyanine tetrasulfonic acid is an intermediate in a route to dyes of Formula (1) it may be chlorinated by reacting with any suitable chlorinating agent.

Chlorination is preferably carried out by treating the phthalocyanine tetrasulfonic acid with chlorosulfonic acid preferably in the presence of an acid halide such as thionyl chloride, sulfuryl chloride, phosphorous pentachloride, phosphorous oxychloride and phosphorous trichloride.

The compounds of Formula (1) have attractive, strong cyan shades and are valuable colorants for use in the preparation of ink-jet printing inks. They benefit from a good balance of solubility, storage stability and fastness to water, ozone and light. In particular they display excellent wet fastness and ozone fastness.

According to a second aspect of the present invention there is provided a composition comprising a mixture of compounds of Formula (1) as described in the first aspect of the invention and a liquid medium.

Preferred compositions according to the second aspect of the invention comprise:
(a) from 0.01 to 30 parts of a mixture of compounds of Formula (1) according to the first aspect of the invention; and
(b) from 70 to 99.99 parts of a liquid medium; wherein all parts are by weight.

Preferably the number of parts of (a)+(b)=100.

The number of parts of component (a) is preferably from 0.1 to 20, more preferably from 0.5 to 15, and especially from 1 to 5 parts. The number of parts of component (b) is preferably from 80 to 99.9, more preferably from 85 to 99.5 and especially from 95 to 99 parts.

Preferably component (a) is completely dissolved in component (b). Preferably component (a) has a solubility in component (b) at 20° C. of at least 10%. This allows the preparation of liquid dye concentrates that may be used to prepare more dilute inks and reduces the chance of the dye precipitating if evaporation of the liquid medium occurs during storage.

The inks may be incorporated in an ink-jet printer as a high concentration cyan ink, a low concentration cyan ink or both a high concentration and a low concentration ink. In the latter case this can lead to improvements in the resolution and quality of printed images. Thus the present invention also provides a composition (preferably an ink) where component (a) is present in an amount of 2.5 to 7 parts, more preferably 2.5 to 5 parts (a high concentration ink) or component (a) is present in an amount of 0.5 to 2.4 parts, more preferably 0.5 to 1.5 parts (a low concentration ink).

Preferred liquid media include water, a mixture of water and organic solvent and organic solvent free from water. Preferably the liquid medium comprises a mixture of water and organic solvent or organic solvent free from water.

When the liquid medium (b) comprises a mixture of water and organic solvent, the weight ratio of water to organic solvent is preferably from 99:1 to 1:99, more preferably from 99:1 to 50:50 and especially from 95:5 to 80:20.

It is preferred that the organic solvent present in the mixture of water and organic solvent is a water-miscible organic solvent or a mixture of such solvents. Examples of such solvents include diethylene glycol, ethylene glycol and 2-pyrollidone. Preferably the liquid medium comprises water and 2 or more, especially from 2 to 8, water-miscible organic solvents.

When the liquid medium comprises organic solvent free from water, (i.e. less than 1% water by weight) the solvent preferably has a boiling point of from 30 to 200° C., more preferably of from 40 to 150° C., especially from 50 to 125° C. The organic solvent may be water-immiscible, water-miscible or a mixture of such solvents.

When the liquid medium comprises a water-immiscible organic solvent, preferably a polar solvent is included because this can enhance the solubility of the mixture of phthalocyanine dyes in the liquid medium.

The organic solvent free from water may be a single organic solvent or a mixture of two or more organic solvents. It is preferred that when the liquid medium is organic solvent free from water it is a mixture of 2 to 5 different organic solvents. This allows a liquid medium to be selected that gives good control over the drying characteristics and storage stability of the ink.

Liquid media comprising organic solvent free from water are particularly useful where fast drying times are required and particularly when printing onto hydrophobic and non-absorbent substrates, for example plastics, metal and glass.

The liquid media may of course contain additional components conventionally used in ink-jet printing inks, for example viscosity and surface tension modifiers, corrosion inhibitors, biocides, kogation reducing additives and surfactants which may be ionic or non-ionic.

Although not usually necessary, further colorants may be added to the ink to modify the shade and performance properties.

It is preferred that the composition according to the invention is ink suitable for use in an ink-jet printer. Ink suitable for use in an ink-jet printer is ink which is able to repeatedly fire through an ink-jet printing head without causing blockage of the fine nozzles. To do this the ink must be particle free, stable (i.e. not precipitate on storage), free from corrosive elements and have a viscosity which allows for good droplet formation at the print head.

A third aspect of the invention provides a process for forming an image on a substrate comprising applying a composition, preferably ink suitable for use in an ink-jet printer, according to the second aspect of the invention, thereto by means of an ink-jet printer.

The ink-jet printer preferably applies the ink to the substrate in the form of droplets that are ejected through a small orifice onto the substrate. Preferred ink-jet printers are piezoelectric ink-jet printers and thermal ink-jet printers. In thermal ink-jet printers, programmed pulses of heat are applied to the ink in a reservoir by means of a resistor adjacent to the orifice, thereby causing the ink to be ejected from the orifice in the form of small droplets directed towards the substrate during relative movement between the substrate and the orifice. In piezoelectric ink-jet printers the oscillation of a small crystal causes ejection of the ink from the orifice. Alternately the ink can be ejected by an electromechanical actuator connected to a moveable paddle or plunger, for example as described in International Patent Application WO00/48938 and International Patent Application WO00/55089.

The substrate is preferably paper, plastic, a textile, metal or glass, more preferably paper, an overhead projector slide or a textile material, especially paper.

Preferred papers are plain or treated papers which may have an acid, alkaline or neutral character. Photographic quality papers are especially preferred.

A fourth aspect of the present invention provides a material preferably paper, plastic, a textile, metal or glass, more preferably paper, an overhead projector slide or a textile material, especially paper more especially plain, coated or treated papers printed with a mixture of compounds as described in the first aspect of the invention, a composition according to the second aspect of the invention or by means of a process according to the third aspect of the invention.

It is especially preferred that the printed material of the fourth aspect of the invention is a print on a photographic quality paper printed using a process according to the third aspect of the invention.

A fifth aspect of the present invention provides an ink-jet printer cartridge comprising a chamber and a composition, preferably ink suitable for use in an ink-jet printer, wherein the composition is in the chamber and the composition is as defined and preferred in the second aspect of the present invention. The cartridge may contain a high concentration ink and a low concentration ink, as described in the second aspect of the invention, in different chambers.

A sixth aspect of the invention provides an amine of Formula (2) and salts thereof:

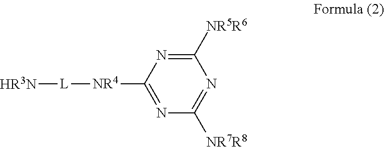

wherein:
R³, R⁴, R⁵ and R⁷ are each independently H or methyl;
R⁶ is H, methyl or aryl carrying 1 to 3 carboxy groups and other optional substituents;
R⁸ is aryl carrying 1 to 3 carboxy groups and other optional substituents; and
L is optionally substituted C₁₋₄alkylene.

Preferably R³, R⁴, R⁵ and R⁷ are all H.

Preferably R⁶ is H, methyl, or phenyl carrying 1 or 2 carboxy groups, more preferably H or methyl and especially H.

Preferably R⁸ is phenyl carrying 1 or 2 carboxy groups.

Preferably the amine of Formula (2) is of Formula (3) and salts thereof:

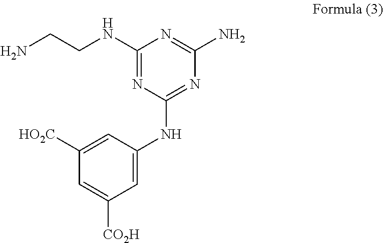

Amines of Formula (2) may be prepared the sequential reaction of cyanuric chloride with amino compounds such as carboxy anilines, ammonia and alkyl diamines using processes which would be well known to one skilled in the art.

The invention is further illustrated by the following Examples in which all parts and percentages are by weight unless otherwise stated.

### EXAMPLES

### Analysis of Compounds of Formula (1)

In all of the Examples below the phthalocyanine is formed by cyclisation of 4-sulfophthalic acid and in all the Examples below it was confirmed by mass spec that the product had 4 substituents (i.e. x+y+z=4). The ratios of x, y and z were determined by elemental analysis. In the results obtained by elemental analysis x plus y plus z is often not exactly 4. A skilled person would not be surprised by this and would know that this discrepancy is due to the presence of impurities. The presence of these impurities and their effect on the estimated values of x, y and z would be well known to a person skilled in the art who would appreciate that the value of x plus y plus z should not exceed 4 and who would treat the experimentally determined values of x, y and z as indicative of the ratios of the groups. Also with some compounds according to the present invention it is not possible using elemental analysis to discriminate between the different sulfonamide substituents. In these cases y and z are quoted as the sum of all the sulfonamide groups which cannot be differentiated e.g. (y+z). Where it was not possible to analyse the compounds formed by elemental analysis the expected values of x, y and z are given.

### Example 1

### Preparation of Amine

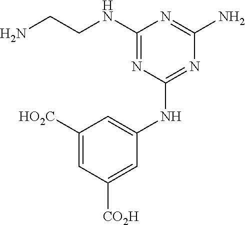

Cyanuric chloride (9.23 g) was stirred in water containing a few drops of calsolene oil at 0-5° C. A solution of 3,5-dicarboxyaniline (9.05 g) in water (75 ml) at pH 7.5 to 8 was then added drop-wise. This reaction mixture was kept below 5° C. and stirred at pH 4 to 5 for 2 hours. The pH was then adjusted to pH 7 with 2M sodium hydroxide, the temperature was raised to 20-25° C. and the reaction mixture was left for 1 hour. Ammonia (9.1 ml) was then added and the pH adjusted to pH 9 to 9.5 (with 2M sodium hydroxide) and the reaction mixture was stirred at room temperature overnight. The next day the reaction mixture was heated to 40° C. for 1 hour, and then ethylenediamine (99 ml) was added and the reaction mixture was heated at 80° C. for a further 2 hours. The reaction mixture was then cooled, sodium chloride was added to give a 20% solution, and the pH was lowered to 1 with concentrated HCl. The precipitate that formed was filtered off and washed with a 20% sodium chloride solution. The precipitate was then slurried in methanol at 60° C., filtered and dried to give the above product (12 g).

### Example 2

### Preparation of a Mixture of Dyes of Formula

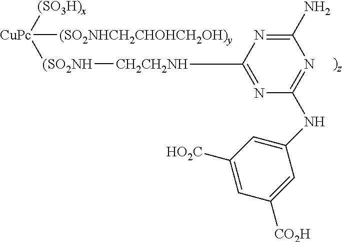

### Stage 1

### Preparation of Copper Phthalocyanine Tetra-β-Sulfonate

Potassium 4-sulfophthalic acid (56.8 g), urea (120 g), CuCl₂ (6.9 g), ammonium molybdate (1.2 g) and 1,8-diazabicyclo[5.4.0]undec-7-ene (DBU) (7.5 g) were mixed in a reaction vessel. The mixture was warmed in stages (130° C./30 minutes, 150° C./30 minutes, 180° C./30 minutes, 220° C./30 minutes) over 2 hours and the melt which formed was stirred at 220° C. for a further 2 hours. The resultant solid was extracted 4 times with hot water (4×200 ml) and the extract was filtered to remove insoluble material. The filtrate was stirred at between 60-70° C. and then sufficient NaCl was added to give a 7% salt solution. Stirring was continued and the precipitate was filtered, washed with a 10% salt solution (200 ml) and pulled dry by vacuum. The resultant damp solid (77.6 g) was slurried in acetone, filtered and dried, first at room temperature and then at 50° C.

### Stage 3

Preparation of the Title Product Phosphorous oxychloride (8.6 g) was added drop-wise to chlorosulfonic acid (86 g) over 5 to 10 minutes while keeping the temperature below 35° C. When all the Phosphorous oxychloride had been added copper phthalocyanine tetra-□-sulfonate, from Stage 1, (16 g) was added portion-wise while keeping the reaction temperature below 55° C. The reaction mixture was stirred at 50-60° C. for 15-20 minutes. The temperature of the reaction mixture was then gradually increased to 138-140° C. over 30 minutes, held at this temperature for 6.5 hours and then the reaction mixture was allowed to cool and stirred overnight at room temperature. The mixture was added to a water/ice (100 ml/150 g) mixture and the resultant precipitate was filtered, washed with ice cold water and filtered. Half the damp filter cake in water (200 ml) was then added to a mixture of the amine from Example 1 (1 molar equivalent, 3.2 g), in water (50 ml) followed by three molar equivalent of 2,3-dihydroxypropylamine (1.99 g) at 0-5° C. The resultant mixture was stirred at 0-5° C. and pH 9 to 9.5 for 1 hour maintaining the pH by the addition of 2M sodium hydroxide. The reaction mixture was then stirred at room temperature overnight. The next day the reaction mixture was heated to 60° C., held at this temperature for 1 hour and then cooled to 40° C. The reaction mixture was then salted with a 20% sodium chloride solution and the pH was lowered to pH 1 with concentrated HCl. The solid that precipitated was filtered, washed with a 20% solution of sodium chloride, dissolved in deionised water, dialysed, filtered and then dried at 70° C. to give the product. Analysis suggested that x was 3.0 and y plus z was 0.2.

### Example 3

### Preparation of a Mixture of Dyes of Formula

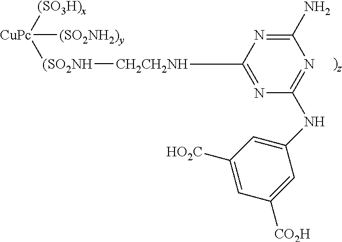

### Preparation of the Title Product

Phosphorous oxychloride (8.6 g) was added drop-wise to chlorosulfonic acid (86 g) over 5 to 10 minutes while keeping the temperature below 35° C. When all the Phosphorous oxychloride had been added copper phthalocyanine tetra-β-sulfonate, from Stage 1, (16 g) was added portion-wise while keeping the reaction temperature below 55° C. The reaction mixture was stirred at 50-60° C. for 15-20 minutes. The temperature of the reaction mixture was then gradually increased to 138-140° C. over 30 minutes, held at this temperature for 6.5 hours and then the reaction mixture was allowed to cool and stirred overnight at room temperature. The mixture was added to a water/ice (100 ml/150 g) mixture and the resultant precipitate was filtered, washed with ice cold water and filtered. Half the damp filter cake in water (200 ml) was then added to a mixture of the amine from Example 1 (1 molar equivalent, 3.2 g), in water (50 ml) at 0-5° C. The resultant mixture was stirred at 0-5° C. at pH 9.5 for 1 hour maintaining the pH by the addition of 28% ammonia. The reaction mixture was then stirred at room temperature overnight. The next day the reaction mixture was heated to 60° C., held at this temperature for 1 hour and then cooled to 40° C. The reaction mixture was then salted with a 20% sodium chloride solution and the pH was lowered to pH 1 with concentrated HCl. The solid that precipitated was filtered, washed with a 20% solution of sodium chloride, dissolved in deionised water, dialysed, filtered and then dried at 70° C. to give the product. Analysis suggested that x was 1.4, y was 1.1 and z was 1.1.

### Comparative Dye

The comparative dye was:

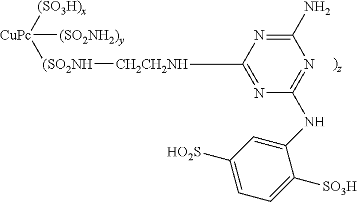

and was prepared as described in Example 3 except that in the preparation of the amine of Example 1, 2,5-disulfoaniline was used in place of 3,5-dicarboxyaniline. Analysis suggested that x was 1.5, y was 1.8 and z was 0.6.

### Preparation of Inks

Inks was prepared by dissolving 3 parts by weight of the dye of Example 3 and the Comparative Dye in 97 parts by weight of a liquid medium comprising % by weight:
Diethylene glycol 7%
Ethylene glycol 7%
2-Pyrollidone 7%
Surfynol™ 465 1%
Tris buffer 0.2%
Water 77.8% and adjusting the pH of the ink to 8-8.5 using sodium hydroxide.
Surfynol® 465 is a surfactant from Air Products.

### Ink-Jet Printing

The Example Ink and Comparative Ink, prepared as described above, were filtered through a 0.45 micron nylon filter and then incorporated into empty print cartridges using a syringe.

This ink was printed as a series of 4 parallel bars at 100% depth, using an ink-jet printer, on to the following ink-jet media.

HP Printing Paper with ColorLok® (HPPC)

HP all-in-one Printing Paper with ColorLok® (HAPPC)

The average reflected optical density and CIE colour co-ordinates L, a, b of the printed bar 4 were then measured using a Gretag® spectrolino spectrophotometer. Readings were taken at 5 sites along the bar and averaged.

### Wet Fastness

The prints obtained above were allowed to dry for 24 hours and evaluated for their wet fastness as follows.

Each print was placed on an A frame at a 45° angle such that the parallel bars were in a horizontal direction. Using an Eppendorf Research Pro electronic pipette 0.1 ml of deionised water was allowed to run down the print. To apply the water to the print the pipette aspirate and dispense function was set to 10 and the pipette was held above the print at an angle of 90° to the lab bench and 5 mm above the top bar of the print. The water was then dispensed down the print and this process was repeated 5 times for each print. The prints were then allowed to air dry for 24 hours. When dry the average reflected optical density and CIE colour co-ordinates L, a, b of printed bar 4 was measured using a Gretag® spectrolino spectrophotometer. Readings were taken at 5 sites along the bar and averaged.

The percentage change in optical density was expressed as % OD where a lower figure indicates higher wet fastness.

The change in shade of printed bar 4 on exposure to water is expressed as ΔE where a lower figure indicates higher wet fastness.

ΔE is defined as the overall change in the CIE colour co-ordinates L, a, b of printed bar 4 of the print and is expressed by the equation

ΔE=(ΔL²+Δa²+Δb²)^{0.5}

where ΔL, Δa and Δb represent the change in the CIE colour co-ordinates after exposure to water.

The Gretag® spectrolino spectrophotometer was set to the following parameters:

Measuring Geometry 0°/45°

Spectral Range 380-730 nm

Spectral Interval 10 nm

Illuminant D50

Observer 2° (CIE 1931)

Density Ansi A

External Filler None

The results of the wet fastness tests are shown in the Tables below.

| | ΔE HPPC | ΔE HAPPC |
|---|---|---|
| Comparative Ink | 4 | 7 |
| Example Ink | 3 | 3 |

| | % OD HPPC | % OD HAPPC |
|---|---|---|
| Comparative Ink | 7 | 15 |
| Example Ink | −6 | −5 |

Clearly prints formed using the compounds and inks of the present invention display an enhanced wet-fastness.

1. A mixture of compounds of Formula (1) and salts thereof: 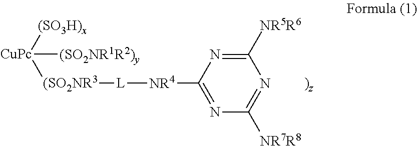
wherein:
Pc represents a phthalocyanine nucleus of formula 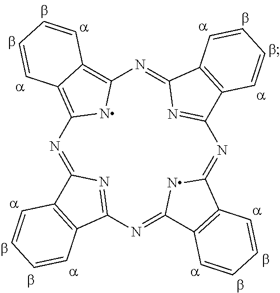
R¹ is H or optionally substituted C₁₋₈alkyl;
R², R³, R⁴, R⁵ and R⁷ are each independently H or methyl;
R⁶ is H, methyl or aryl carrying 1 to 3 carboxy groups and optionally other substituents;
R⁸ is aryl carrying 1 to 3 carboxy groups and optionally other substituents;
L is optionally substituted C₁₋₄alkylene;
x, y, and z are each independently greater than 0 and less than 4;
x+y+z is in the range of 2 to 4; and
provided that:
the substituents represented by x, y and z are attached to the phthalocyanine nucleus by a β-position; and
L, R′, R⁶ and R⁸ do not carry a sulfonic acid substituent.

2. A mixture of compounds and salts thereof as claimed in claim 1 wherein R¹ is H or methyl.

3. A mixture of compounds and salts thereof as claimed in claim 1 wherein R¹ is optionally substituted C₁₋₄alkyl carrying at least one water solubilising substituent selected from the group consisting of: —OH, —CO₂H and —PO₃H₂.

4. A mixture of compounds and salts thereof as claimed in claim 1 wherein R¹ is C₁₋₄alkyl with a phenyl substituent carrying 1 or 2 water solubilising groups selected from the group consisting of —CO₂H and —PO₃H₂.

5. A mixture of compounds and salts thereof as claimed in claim 1 wherein R¹ is C₁₋₈alkyl substituted with 2 or more —OH groups and optionally other substituents.

6. A mixture of compounds and salts thereof as claimed in claim 1 wherein R⁶ is H, methyl or optionally substituted phenyl carrying 1 or 2 carboxy groups.

7. A mixture of compounds and salts thereof as claimed in claim 1 wherein R⁸ is optionally substituted phenyl carrying 1 or 2 carboxy groups.

8. A mixture of compounds and salts thereof as claimed in claim 1 wherein L is —C₂H₄—.

9. A mixture of compounds and salts thereof as claimed in claim 1 wherein x is greater than 1.

10. A mixture of compounds and salts thereof as claimed in claim 1 wherein the sum of (x+y+z) is 4.

11. A composition comprising a mixture of compounds of Formula (1) as described in claim 1 and a liquid medium.

12. A process for forming an image on a substrate comprising applying a composition according to claim 11 thereto by means of an ink-jet printer.

13. A material printed with a mixture of compounds as described in claim 1.

14. An ink-jet printer cartridge comprising a chamber and a composition wherein the composition is in the chamber and the composition is as defined in claim 11.

15. An amine of Formula (2) and salts thereof: 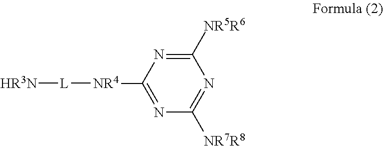
wherein:
R³, R⁴, R⁵ and R⁷ are each independently H or methyl;
R⁶ is H, methyl or aryl carrying 1 to 3 carboxy groups and other optional substituents;
R⁸ is aryl carrying 1 to 3 carboxy groups and other optional substituents; and
L is optionally substituted C₁₋₄alkylene.
